(19) United States
(12) Patent Application Publication
(10) Pub. No.: US 2010/0280454 A1
(43) Pub. Date: Nov. 4, 2010

(54) METHOD AND APPARATUS FOR WARMING OR COOLING A FLUID

(76) Inventor: Keith Michael Rosiello, Shrewsbury, MA (US)

Correspondence Address: FOLEY & LARDNER LLP, 111 HUNTINGTON AVENUE, 26TH FLOOR, BOSTON, MA, 02199-7610, US

(21) Appl. No.: 12/523,837

(22) PCT Filed: Jul. 25, 2007
(86) PCT No.: PCT/US07/16762

§ 371 Date: Jun. 8, 2010

Related U.S. Application Data
(60) Provisional application No. 60/916,527, filed on May 7, 2007.

(51) Int. Cl. A61M 5/44

(52) U.S. Cl. 604/114

(57) ABSTRACT

A method and system for heating or cooling a fluid to be delivered into the body of a patient is provided and may include a controller and a fluid delivery line assembly. The fluid delivery line may include sterile fluid pathway and for communicating a fluid from a source to a destination, one or more terminating connectors, and an integral resistance element for producing heat in response to electrical current. The fluid delivery line may also include one or more thermal sensor positioned within the fluid pathway for detecting and reporting the temperature of the fluid being delivered. The controller may include an embedded control/feedback logic program with novel heat balance algorithm to precisely control the amount of heat applied to the fluid. The fluid delivery line assembly may be disposable with at least one of the thermal sensors being reusable while maintaining sterility within the fluid pathway.

### RELATED APPLICATIONS

This application claims the benefit of U.S. Provisional Application No. 60/916,527, entitled “Method and System for Warming a Fluid,” filed on May 7, 2007. This application is also a Continuation-in-part of International Application Ser. No. PCT/US2007/001510, entitled “Method and System for Warming or Cooling a Fluid” filed in the U.S. Receiving Office under the Patent Cooperation Treaty on Jan. 19, 2007, which itself claims the benefit of U.S. Provisional Application Nos. 60/760,771 entitled “Method and System for Warming or Cooling a Fluid,” filed on Jan. 19, 2006, the entire teachings of all of which are incorporated herein by reference.

### FIELD OF THE INVENTION

The invention is directed generally to a method and apparatus for warming or cooling a fluid, and more particularly, to a method and system for warming or cooling a fluid to be delivered into the body of a patient.

### BACKGROUND OF THE INVENTION

Most parenteral fluids, such as saline, are commonly stored at “normal room temperature” generally considered 65° F.-75° F. (18.3° C.-23.9° C.). Other parenteral fluids, such as whole blood, are stored at the refrigerated temperature of 39.2° F. (4° C.). Yet other parenteral fluids are cryo-preserved and, due to time constraints, often only uniformly thawed just enough to allow fluid flow. It is advantageous for intravenously administered parenteral fluids to be warmed to near normal body temperature to prevent insult to the patient and, in hypothermia related cases, reduce the level of trauma.

A number of apparatuses and methods have been designed to address the need to warm parenteral fluids for use in transfusion medicine. Most common are bulk fluid warmers. These devices warm a bulk volume of fluid such as a bag of whole blood using a reservoir of heated fluid, the fluid usually being water. The bag of fluid to be warmed is doubled bagged for safety and immersed in the heated bath while being constantly mixed to insure uniform heating. After some time, usually 10-40 minutes, depending on the starting and desired fluid temperatures, the fluid is ready to be transfused.

Other prior art devices include in-line warmers, which are used to warm fluids for use in transfusion medicine. These devices use various heating techniques to warm fluids as they flow from the supply bag to the patient. The heating techniques vary greatly as shown in the cited prior art. For example, U.S. Pat. No. 5,690,614 uses microwave energy, U.S. Pat. No. 5,807,332 uses a heated stream of air, and U.S. Pat. No. 5,101,804 uses a chemical reaction. Other prior art references use electrically heated plates in either direct or indirect contact with the fluid to be warmed.

The methods and apparatus available to suitably warm fluids have several limitations in common. The primary limitation shared by a majority of current fluid warmers, or blood warmers, as they are sometimes known, is an inability to achieve the target fluid temperature at low flow rates, which results from the need to protect the fluid from overheating. The inability of these current fluid warmers to achieve optimal performance at low flow rate is due to the lack of sensitivity and fine control in the regulation of the heat supplied to the fluid. Inefficient application of heat to the fluid and extended, serpentine flow paths between fluid supply and patient negatively effect the response-time of many fluid warmers, hence a large safety factor is required at low flow rates to reduce the probability of overheating the fluid.

Another limitation of current fluid warmers is the lack of flexibility with respect to flow rate range, fluid temperature output and physical specification, due to the singular focus of these methods and apparatus on fluid warming in a surgical environment. Current fluid warmers do not posses the sensitivity and rapid response-time required for optimal performance in emergent applications, such as ambulances, emergency rooms and field use. For example, the use of higher flow rates and the occurrences drastic flow rate changes are rarely encounter in surgical situations, but may often be necessary in emergent situation. The inefficient heat-application methods used by current fluid warmers dramatically increase the response-time, which results in a failure to reach the target fluid temperature a high flow rates. Similarly, the extended flow path and lack of sensitivity in current fluid warmers results in fluid overheating during a drastic flow rate reduction, which is rare event in a surgical environment.

In addition, much of the prior art is designed to be a modular component within the total intravenous administration set. This often requires the use of a pre-warmer IV set and a post-warmer IV set. These IV sets may need to be several feet long to accommodate the spatial logistics of a surgical procedure or the chaos of an emergency room. The post-warmer IV set is a source of significant heat loss, creating a varying temperature differential between the fluid warmer and the patient. Furthermore, the need for IV sets is not preferred for portability and field use.

Finally, the current methods and apparatus available to suitably warm fluids are all comprised of at least one reusable unit and one disposable unit, which function jointly to add heat to the fluid for transfusion. The reusable unit provides power control and heat generation, while the disposable unit provides a sterile, single-use chamber or pathway to accommodate the fluid for transfusion during the application of heat. The majority of the daily operating costs of current warmers are attributed to the cost of the disposable unit. However, the cost of the disposable unit for current fluid warmers can be high, due to the inclusion of multiple high-value components and the use complex manufacturing processes.

### SUMMARY OF THE INVENTION

Accordingly, the present invention overcomes the above-noted problems and concerns, and some embodiments of the present invention provide a novel fluid warmer for delivering a fluid, medicinal or otherwise, into the body of a patient. Patient may include any living organism, especially mammal, and humans in particular.

The invention described herein overcomes the aforementioned limitations by integrating the IV set with a novel warming method and apparatus, for example. In one embodiment of the present invention, the novel method and apparatus may use a variety of power sources from AC to a small battery of both rechargeable and disposable types. The method and apparatus may also include a single delivery-line unit (disposable unit) between the fluid supply bag and the patient connection to warm the fluid along its entire length by integrating the fluid delivery tube with a heat source. The method and apparatus may also include a controller (reusable unit), which contains and executes control/feedback logic program with unique heat balance algorithm that provides a precise level of control, in a full range of transfusion environments, on the amount of heat applied to the fluid. The method and apparatus may also include a single delivery-line component with minimal manufacturing cost.

Accordingly, this novel design according to some embodiments of the present invention may allow the fluid delivery pathway to be flexible, non-kinking, in lengths of 1 foot and greater. The choice of power sources, heat balance algorithm, and the ability of the fluid warmer to act as an IV set enables some of the embodiments of the present invention well suited to portability and use in a variety of environments. Gradual and efficient warming over the entire non-serpentine fluid delivery length, for example, may support low and high (1 mL/min to 600 mL/min) flow rates for a variety of parenteral fluids, including whole blood, substantially eliminating or limiting damage to the fluid and/or patient, or overheating.

Thus, the new design according to some embodiments of the present invention may provide a fluid warmer that is portable, adaptable to different environments and easy to use.

Accordingly, in one embodiment of the present invention, a method of heating a fluid for delivery into the body of a patient may include providing a low cost fluid delivery tube having a first end for connection to a fluid source and a second end for delivering the fluid from the fluid source to a destination. The method may also include applying an electrical current to a heating element proximate to and/or within the fluid delivery tube to heat fluid therein to a predetermined temperature, sensing, via multiple thermal sensor positioned on the fluid delivery tube and in the fluid pathway, fluid temperatures. Then, the fluid temperature values are used by the heat balance algorithm to adjust the current applied to heating element, which regulates the amount of heat applied to the fluid.

In another aspect, the invention relates to a disposable assembly for heating or cooling a fluid flow destined for delivery into a body of a patient. The disposable assembly includes an elongated fluid transfer tube including a lumen extending therethrough and a distal connector assembly attached at one end of the elongated fluid transfer tube. The distal connector assembly includes a fluid channel in fluid communication with the lumen of the elongated fluid transfer tube. The distal connector assembly also includes a fitting for connecting the fluid channel to a fluid inlet tube, and an axial shroud. The axial shroud is open at one end and extends at least partially into the fluid path. The shroud is adapted to receive a thermal probe inserted at the open end and isolating the thermal probe from fluid path.

In another aspect, the invention relates to a system for heating a fluid for delivery into a body of a patient. The system includes an elongated fluid delivery line including a tube for communicating a fluid along a fluid path. The elongated fluid delivery line also includes at least one fluid connector coupled to one end of the fluid delivery line and at least one elongated fluid-tight shroud open at one end and extending at least partially into the fluid path. The shroud is adapted to receive a thermal probe at the open end.

In yet another aspect, the invention relates to a process of heating or cooling a fluid for delivery into the body of a patient. The process includes providing an elongated fluid delivery tube having a first end adapted for connection to a fluid source and a second end adapted for delivering the fluid from the fluid source to a destination. An electrical current is applied to a heating or cooling element proximate to and/or within the elongated fluid delivery tube to heat fluid therein to a predetermined temperature. A reusable thermal sensor is positioned within a disposable fluid-tight shroud, which extends into a fluid path at one end of the fluid delivery tube. A temperature corresponding to the temperature of the fluid within the tube is sensed using the reusable thermal sensor. The current applied to heating element is adjusted based upon the sensed temperature to cause a change in temperature.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is an illustration of an overall fluid heat-transfer system according to some embodiments of the present invention.

FIG. 2 is a schematic diagram of a portion of a fluid heat-transfer system according to some embodiments of the present invention.

FIG. 3 is an electrical schematic diagram of a fluid delivery line of the type shown in FIG. 2.

FIG. 4A is a side view of a patient-distal, fluid-delivery line termination assembly according to some embodiments of the present invention.

FIG. 4B and FIG. 4C are respectively front and rear perspective views of the fluid-delivery line termination assembly shown in FIG. 4A.

FIG. 5A is a central cross-sectional view of the fluid-delivery line termination assembly shown in FIG. 4A.

FIG. 5B and FIG. 5C are respectively front and rear perspective exploded views of the fluid-delivery line termination assembly shown in FIG. 4A.

FIG. 6 is a front perspective view of a controller box adapter module according to some embodiments of the present invention.

FIG. 7A is a central cross-sectional view of the controller box adapter module shown in FIG. 6.

FIG. 7B is a non-central cross-sectional view of the controller box adapter module shown in FIG. 6.

FIG. 8A and FIG. 8B are respectively front and rear perspective views of the controller box adapter module shown in FIG. 6.

FIG. 9A(1), FIG. 9B(1), FIG. 9C(1), FIG. 9D(1), and FIG. 9F are top perspective views illustrating an alignment and interconnection sequence of the fluid-delivery line termination assembly shown in FIG. 4A with the controller box adapter module shown in FIG. 6.

FIG. 9A(2), FIG. 9B(2), FIG. 9C(2), FIG. 9D(2) are cross-sectional elevation views and FIG. 9E a top perspective cross-sectional view, together illustrating the alignment and interconnection sequence of the fluid-delivery line termination assembly shown in FIG. 4A with the controller box adapter module shown in FIG. 6.

FIG. 10 is an exploded view of a patient-proximal luer lock assembly according to some embodiments of the present invention.

FIG. 11A is a side view of the luer lock assembly shown in FIG. 10.

FIG. 11B and FIG. 11C are respectively front and rear perspective views of the luer lock assembly shown in FIG. 10.

FIG. 12 is a cross-sectional elevation view of the fluid-delivery line termination assembly shown in FIG. 4A being coupled to an alternative embodiment of a controller box adapter module according the present invention.

FIG. 13A is a detailed-view diagram illustrating one embodiment of a main heat tube component of a fluid delivery line for use in a fluid heat-transfer system.

FIG. 13B is a is a cross section diagram illustrating a cross section of the main heat tube component of a fluid delivery line for use in a fluid heat-transfer system.

FIG. 14 is a cross section diagram illustrating a cross-section of a fluid delivery-line for use in a fluid warming system according to some embodiments of the present invention.

FIG. 15 is a cross-sectional diagram illustrating a cross-section of a fluid delivery line for use in a fluid heat-transfer system according to some embodiments of the present invention.

FIG. 16 is a top perspective view of a controller module for use in a fluid heat-transfer system according to some embodiments of the present invention.

FIG. 17A and FIG. 17B are functional block diagrams of fluid heat-transfer systems according to some embodiments of the present invention.

FIG. 18 is a flow diagram illustrating functionality of an embedded control/feedback logic program of a controller for use in a fluid heat-transfer system according to some embodiments of the present invention

FIG. 19A and FIG. 19B are flow diagrams illustrating in more detail functionality of an embedded control/feedback logic program of a controller for use in a fluid heat-transfer system according to some embodiments of the present invention.

### DETAILED DESCRIPTION OF THE EMBODIMENTS

A description of preferred embodiments of the invention follows.

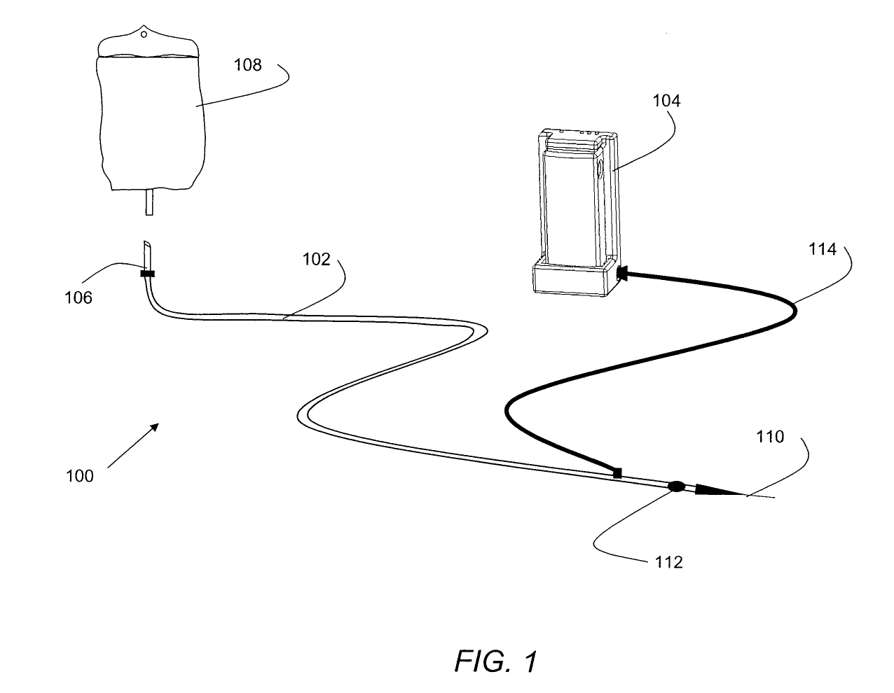

As shown in FIG. 1, some of the embodiments of the present invention include the following features. A fluid warming system 100 may include a fluid delivery line 102 and a controller 104. The system may further include a lead tube 106, comprised of standard medical grade tubing, one end of which is attached and sealed to the control end of the fluid delivery line, using commonly know techniques such as solvent bonding or barbed tube fittings. A luer lock 108 may be connected to the other end of the lead tube, and may be used to fluidly connect the fluid delivery line to a container 112 (e.g., bag) of fluid for delivery to the body of a patient. Such luer locks may include those disclosed in U.S. Pat. Nos. 5,620,427, 5,738,144 and 6,083,194, each of which is herein incorporated by reference. Each of the luer locks is preferably attached to the fluid delivery line and forms a sterile and/or airtight seal thereto. Each luer lock may include a standard cap component, for the protection of the sterile and/or airtight fluid delivery line. In addition to the luer locks, bag spikes may be incorporated at an end of the lead tube. Such bag spikes may include, for example, U.S. Pat. Nos. 5,445,630, 4,432,765 and 5,232,109, herein incorporated by reference.

A luer lock 117 may be connected at the other (patient) end of the fluid delivery line to provide a standard means of attachment for a transfusion needle, for example. The transfusion needle, or similar device, is inserted into, for example, a blood vessel of the patient, so that the fluid traversing through the fluid delivery line may enter the body of the patient. In addition to the luer locks, transfusion needles or the like may also be directly incorporated at an of the fluid delivery line. Each of the luer locks is preferably attached to the fluid delivery line and forms a sterile and/or airtight seal thereto. Each luer lock may include a standard cap component, for the protection of the sterile and/or airtight fluid delivery line. Moreover, in some embodiments, it is preferable that the fluid delivery line be sterile or sterilized prior to use. In one embodiment of the present invention, the fluid delivery line with luer locks and connection lines (preferably all together; the “fluid delivery line system”), is a single use system, which is sterilized upon manufacture and sealed in an airtight package. When used, the package is opened and the system (or individual components) is connected to the fluid container and controller and used for delivering the fluid contained in the container to the body of a patient. After this single use, the fluid delivery line system is disposed, preferably as medical waste.

A valve may be positioned along the fluid delivery line at any position, for controlling the flow of the fluid within the inner fluid delivery tube. In one embodiment, the valve is positioned adjacent to the patient end of the fluid delivery line system. In this embodiment, the valve may be mechanically and/or electrically actuated by the controller, individual (e.g., medical personnel), or may be a passively operated valve which may be actuated by a change in temperature of the fluid within the fluid delivery tube. In that regard, the valve may be made of a bi-metal material which opens upon the temperature of the fluid reaching predetermined temperature. In another embodiment, the controller may provide an electrical actuation signal for the valve to open when, for example, the thermal sensor positioned at the patient end of the fluid delivery line system indicates the desired fluid temperature is reached. In such an embodiment, the valve may also be positioned near the transfusion needle to control the flow of fluid from the fluid delivery line into the patient.

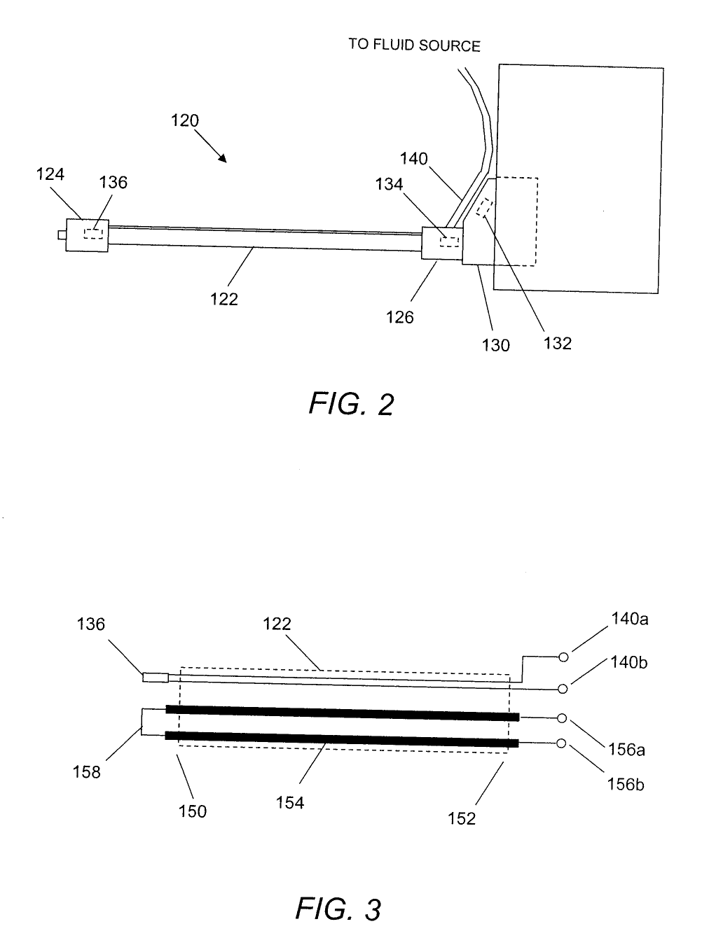

As shown in FIG. 2, a schematic diagram of a portion of a fluid heat-transfer system includes a fluid delivery tube assembly 120 disposed between a fluid source or reservoir 108 (FIG. 1) and a patient (not shown). The fluid delivery tube assembly 120 includes a thermal transfer fluid delivery tube 122 having at one end a patient-proximal luer lock assembly 124 and at an opposite end a patient-distal, control end termination 126. The control end termination 126 is coupled to the reservoir 108 through a fluid inlet tube 140 and to a controller 128 through a controller box adapter module 130.

The fluid delivery tube assembly 120 includes a fluid inlet temperature sensor 132 sensing a temperature of fluid from the reservoir 108 prior to entering the fluid delivery tube assembly 120. The fluid delivery tube assembly 120 also includes a flow-out temperature sensor 136 positioned at a patient-proximal end of the delivery tube assembly 120. Temperature sensor lead wires 138 run along the fluid delivery tube, 122 from the flow-out temperature sensor 136 to the control end termination 126. Alternatively or in addition, one or more additional temperature sensors can be provided at different positions along the thermal transfer fluid delivery tube 122. In some embodiments, the fluid delivery tube assembly 120 is a disposable item including the fluid delivery tube 122, the proximal luer lock 124, the flow-out temperature sensor 136, the temperature sensor lead wires 138 and the control end termination 126. As will be described in more detail below, a flow-in temperature sensor 134 is provided at a patient-distal end of the fluid delivery tube 122, in the general vicinity of the control end termination 126.

An electrical schematic diagram of an exemplary fluid delivery tube 122 is shown in FIG. 3. One or more elongated thermal transfer elements are run along a substantial length of the fluid delivery tube 122. For example, a pair of resistive heating elements 154 is run along the length of the fluid delivery tube 122. An electrical interconnect, or jumper 158 is included at the patient-proximal end 150 of the fluid delivery tube 122, between the pair of resistive heating elements 154. Electrical terminals 156a, 156b located at the patient-distal end 152 of the resistive heating elements 154 can be coupled to an electrical power source to provide a current within the heating elements 154 to heat a fluid contained within the fluid delivery tube 122.

The flow-out temperature sensor 136 can be an electrical temperature sensor, such as a thermocouple. The flow-out temperature sensor 136 can be positioned adjacent to or at least partially within the fluid flow of the fluid delivery tube 122. An electrical potential representative of temperature is measurable between electrical terminals 140a, 140b at the patient-distal end 152 of the temperature sensor lead wires 138. Thermal potential at the junction of the thermocouple produces a potential across the terminals 140a, 140b that is proportional to a temperature of the fluid at the patient-proximal end 150 of the fluid delivery tube 122. The controller 128 (FIG. 2) as described in more detail below is configured to energize the resistive heating elements 154, to measure a potential across the temperature sensor lead wire terminals 140a, 140b, and to calculate a flow-out temperature based on the measured potential.

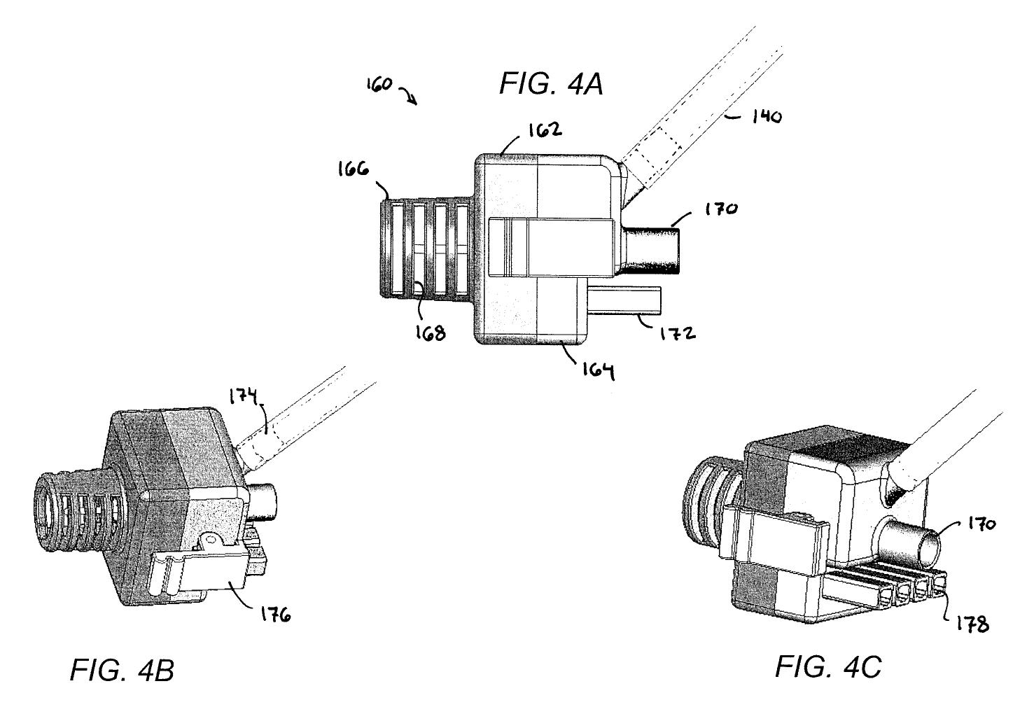

Referring now to FIG. 4A, FIG. 4B, and FIG. 4C, an exemplary embodiment of the control end termination 160 is illustrated in more detail. The control end termination 160 includes a fluid delivery line member 162 and a box adapter member 164. The fluid delivery line member 162 includes a tubing support collar 166 for supporting a patient-distal end of the fluid delivery tube 122. In particular, the tubing support collar 166 prevents bending of the tube in the vicinity of the control end termination 160. In some embodiments, the tubing support collar 166 includes an axial array of circumferential slots 168. The slots reduce the interior surface area of the tubing support collar 166 in contact with an exterior surface of the fluid delivery tube 122, thereby reducing sliding friction. This allows for a snug fit between the collar 166 and the tube 122, while allowing the collar to slide along the delivery tube 122 during an assembly procedure. In some embodiments, the control end termination 160 assembly is formed using no more than two injection molded parts that snap together protecting all electrical connections, without the need for heat shrink tubing or lead wires. Ends of the elongated resistive heater elements or wires can be fastened directly to electrical terminals 178 within the control end termination 160 assembly, by crimp or solder, or any other suitable method of electrically conductive attachment.

The box adapter member 164 includes an alignment/support collar 170 and a fluid inlet fitting 174 sized and adapted to form a fluid-tight seal with the fluid inlet tube 140. In an exemplary embodiment, the fluid inlet fitting 174 is a barbed fitting adapted to form a friction fit with an interior surface of a resilient fluid inlet tube 140. The box adapter member 164 also includes an electrical connector plug 172 including one or more electrical terminals 178 therein. Electrical terminals 178 of the connector plug 172 are respectively coupled to the terminals 140a, 140b, 154a, 154b at the patient-distal end of the fluid delivery tube 122 and are usable to electrically interconnect the fluid delivery tube 122 to the controller 128 (FIG. 2). A pair of latches 176 are pivotally coupled to the box adapter member 164 and adapted to engage a mounting surface located on the controller box adapter module 130 (FIG. 2). Each of the latches 176 can be spring loaded and biased in a closed position.

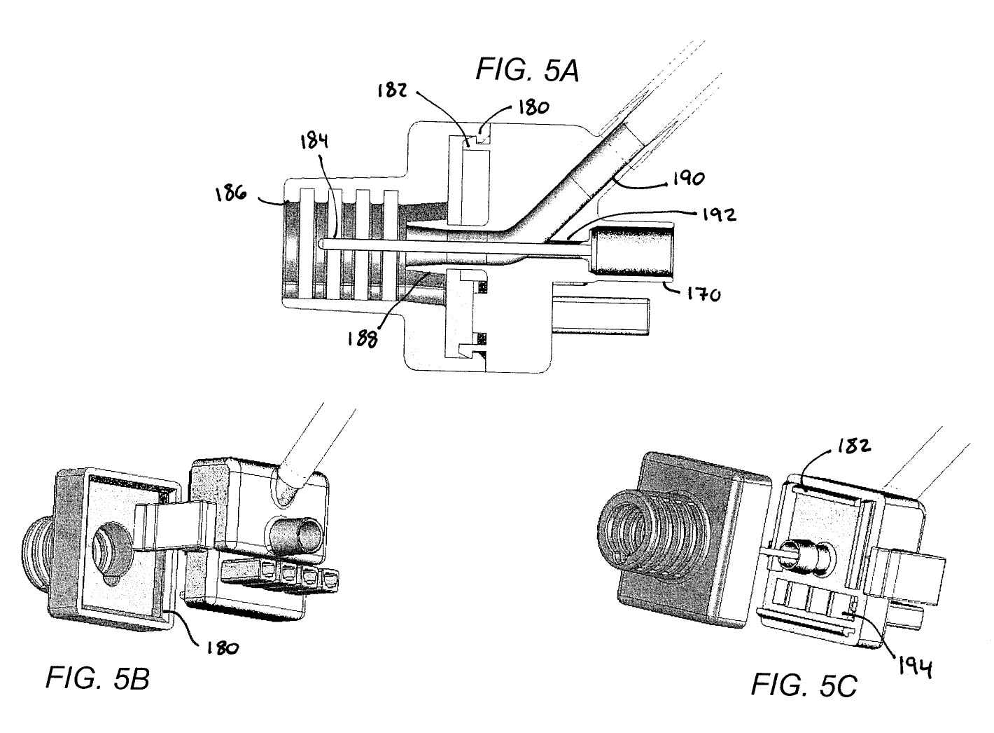

Referring now to FIG. 5A, FIG. 5B, and FIG. 5C, the box adapter member 164 in more detail includes a barbed tubing fitting 188 axially positioned within the tubing support collar 166. The barbed tubing fitting 188 is sized and shaped to fit within an interior lumen of a compliant fluid delivery tube 122, forming a friction fit therewith. The box adapter member 164 also includes a fluid inlet lumen 190 providing fluid communication between the fluid inlet fitting 174 and a patient-distal end of the fluid delivery tube 122 through the barbed tubing fitting 188. A second lumen 192 is positioned between the alignment/support collar 170 and the patient-distal end of the fluid delivery tube 122. An elongated sheath 184 open at one end only is positioned axially, extending from the alignment/support collar 170 and extending into the patient-distal end of the fluid delivery tube 122. A closed end of the elongated sheath 184 extends into the delivery tube 122 and an open end faces the alignment/support collar 170, such that fluid transfer between the fluid channel and the alignment/support collar 170 is prohibited. In the exemplary embodiment, the barbed tubing fitting 188 and the tubing support collar 166 are coaxially disposed with respect to the axial sensor sheath 184. The fluid inlet lumen veers off at an angle, forming a ‘y’ junction with the sensor lumen 192 and the lumen of the barbed tubing fitting 188.

In the exemplary embodiment, the control end termination 160 is formed of two molded parts: the fluid delivery line termination 162 and the box adapter member 164. In some embodiments, the two components 162, 164 are configured to fasten together using a snap fit interconnection. As shown, the fluid delivery line termination 162 includes a retaining ridge 180 extending along an inside perimeter of an adjoining end. The box adapter member 164 includes one or more complementary interlocking fingers 182 positioned along a perimeter of an adjoining end.

Visible along an adjoining surface of the box adapter member 164 is an array of wire access openings 194. Each wire access openings is in communication with a respective electrical contact disposed therein. During an assembly procedure, a patient-distal end of the fluid delivery line 122 is fitted through a central opening of the tubing support collar 166. The fluid delivery line termination 162 is urged along the fluid delivery line 122 away from a patient-distal end of the fluid delivery line 122. Electrical contacts are then established between the one or more electrical terminals 140a, 140b, 154a, 154b of the fluid delivery line 122 and their respective electrical contacts 178. The patient-distal end of the fluid delivery line 122 is then urged onto the barbed tubing fitting 188 forming a secure attachment between the fluid delivery line 122 and the box adapter member 164. The fluid delivery line termination 162 is next urged along the fluid delivery line 122 toward the box adapter member 164 until the interlocking fingers 182 engage with the retaining ridge 180, resulting in an interlocking engagement of the delivery line termination member 162 and the box adapter member 164.

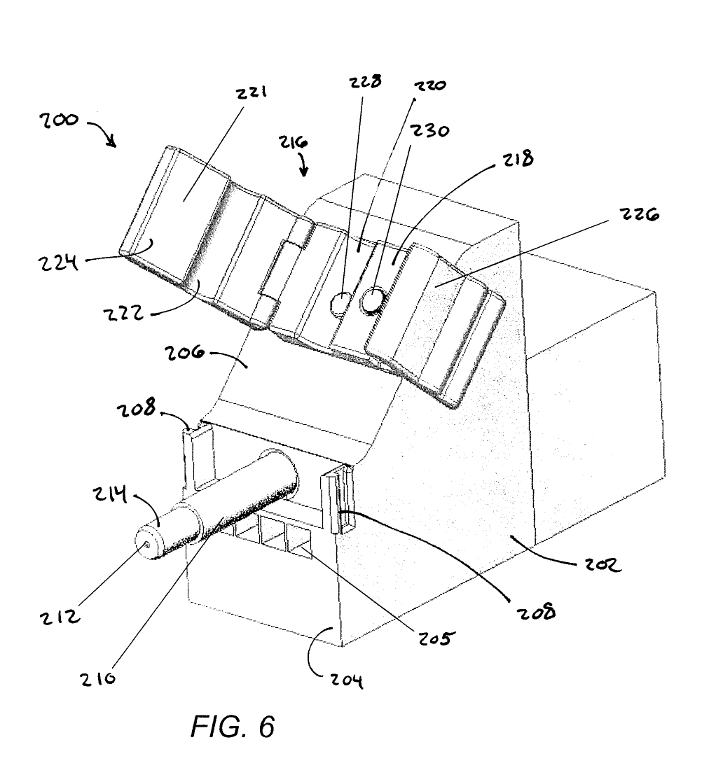

As shown in FIG. 6, a controller box adapter module 200 includes a housing 202 having a fluid delivery line termination abutting surface 204 and a sloped surface 206 extending at a sloping angle with respect to the abutting surface 204. The abutting surface 204 can include one or more electrical connector receptacles 205 positioned for alignment with the one or more electrical connector plugs 172 of the fluid termination assembly 160 (FIG. 4). The housing 202 also includes a pair of mounting surfaces 208 engageable by the pivotal latch 176 (FIG. 4B) to releasably retain the patient-distal fluid delivery tube termination 160 against the fluid delivery tube termination abutting surface 204. A spring loaded retractor pin 210 extends away from the abutting surface 204 and is positioned for axial alignment with and partial insertion within the alignment support collar 170 (FIG. 4) when mated. To facilitate alignment with the alignment support collar 170, a proximal end of the spring loaded retractor pin 210 includes a reduced diameter and/or taper 214. The spring loaded retractor pin 210 also includes an axial bore, such that the retractor pin 210 when fully extended enshrouds a patient-proximal end of a permanent flow-in temperature sensor 212. The flow-in temperature sensor 212 is fixedly mounted with respect to the housing 202. Operation of the spring loaded retractor pin 210 is described in more detail below.

The controller box adapter module 200 also includes a fluid inlet tube retaining assembly 216. The tube retaining assembly 216 can be mounted on the sloped surface 206 for alignment with the fluid inlet tube 140 coupled to the fluid delivery tube termination 160, when the termination 160 is coupled to the housing 202. The tube retaining assembly 216 includes a fixed fluid inlet tube guide 218 that can include a tube alignment saddle 220. A fluid inlet tube retaining door 221 is connected by a hinge to one end of the tube guide 218. The tube retaining door 221 includes a fluid inlet tube notch 272 and an abutting surface 224. A pivotal latch 226 is pivotally attached to an opposite end of the tube guide 218 and adapted to retain the tube retaining door 221 in a closed position, thereby entrapping the fluid inlet tube 140 along the tube alignment saddle 228. In some embodiments, the controller box adapter module 200 is made from no more than five molded parts, four screws, a reusable flow in temperature sensor hard mounted with respect to the housing 202 and a switch 230 to make sure the fluid inlet tube retaining door 221 is closed and locked.

In some embodiments, the tube retaining assembly 216 includes a sensor 230 detecting whether the fluid inlet door 221 is closed. For example, the sensor 230 can be a momentary switch positioned to engage the abutting surface 224 and actuate when the tube retaining door 221 is fully closed. Alternatively or in addition, the tube retaining assembly 216 includes a fluid inlet temperature sensor 228 positioned to measure a temperature of the fluid within the fluid inlet tube 140 (FIG. 4). For example, the fluid inlet temperature sensor 228 includes a thermocouple positioned within the tube alignment saddle 228 for thermal contact with an exterior surface of the fluid inlet tube 140.

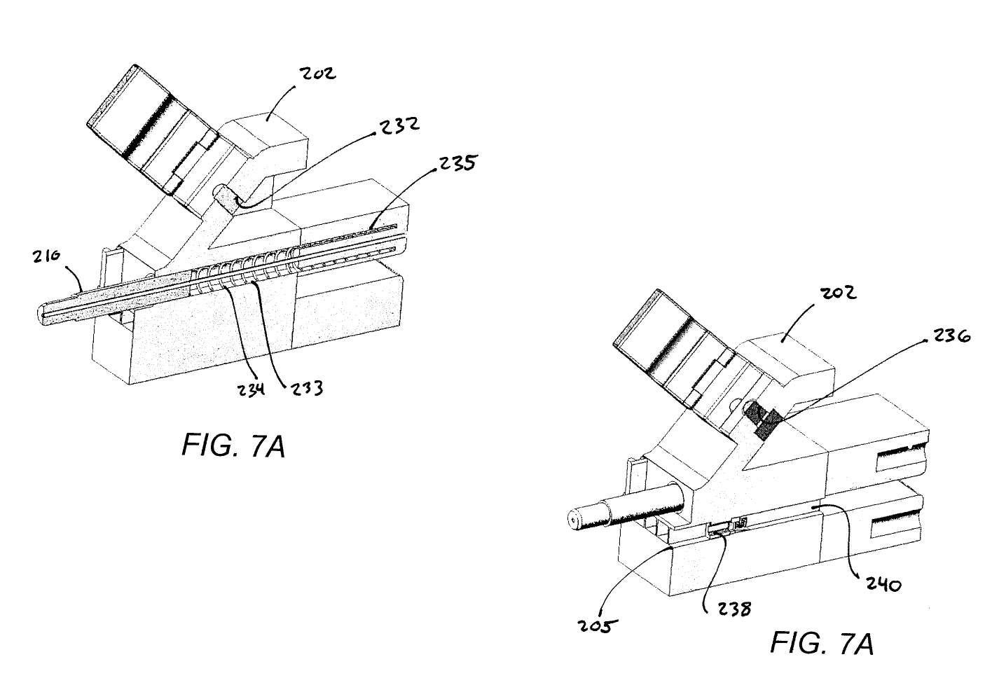

As shown in the central cross-sectional view of the controller box adapter module 200 shown in FIG. 7A, a patient-proximal end of the flow-in temperature sensor 212 is disposed within a fully extended spring loaded retractor pin 210. The retractor pin 210 is urged in the fully extended position by a compression spring 234 disposed within an axial cavity 233 defined in the housing 202. The cavity 233 is dimensioned to accommodate the spring-loaded retractor pin 210 in a retracted position. In some embodiments, a distal end of the compression spring 234 is retained within an annular cavity 235 formed along a distal end of the housing 202. Also visible is an access channel 232 for the fluid inlet temperature sensor 228. A portion of an electrical contact 238 is visible within one of the electrical connector receptacles 205 in the non-central cross-sectional view of FIG. 7B. The contact 238 is adapted for electrical interconnection with a corresponding contact 178 (FIG. 4) of a mating electrical plug connector 172 (FIG. 4). Cable access from the contact 238 to the controller 128 (FIG. 2) is provided by a slot or cable way 240. Also visible is an access channel 236 for the fluid inlet tube door switch 230.

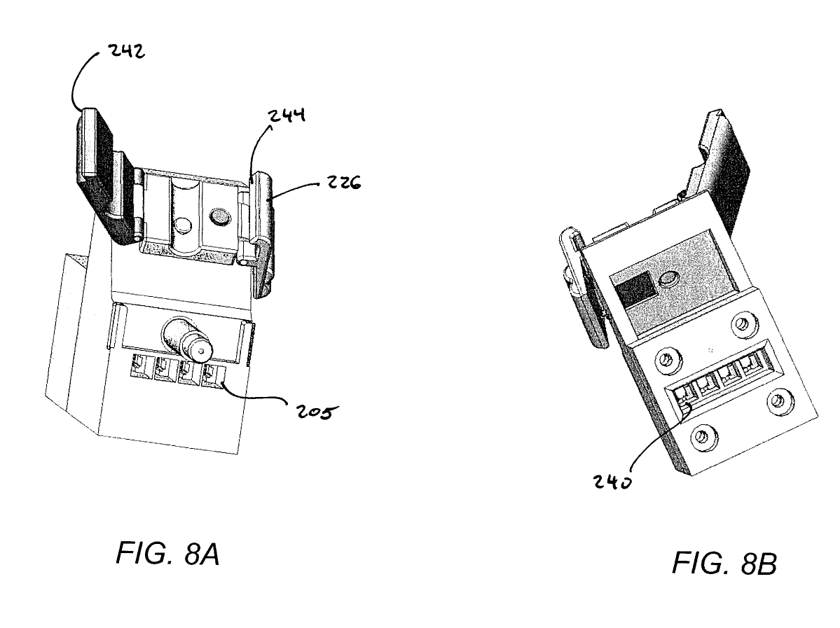

Front and rear perspective views of the controller box adapter module are shown respectively in FIG. 8A and FIG. 8B. The fluid inlet tube retaining assembly 216 is shown in an open position. One end of the tube retaining door 221 includes a retaining surface 242 adapted to engage an engaging end 244 of the pivotal latch 226 when closed. In some embodiments, the pivotal latch 226 is spring loaded and biased in a closed position.

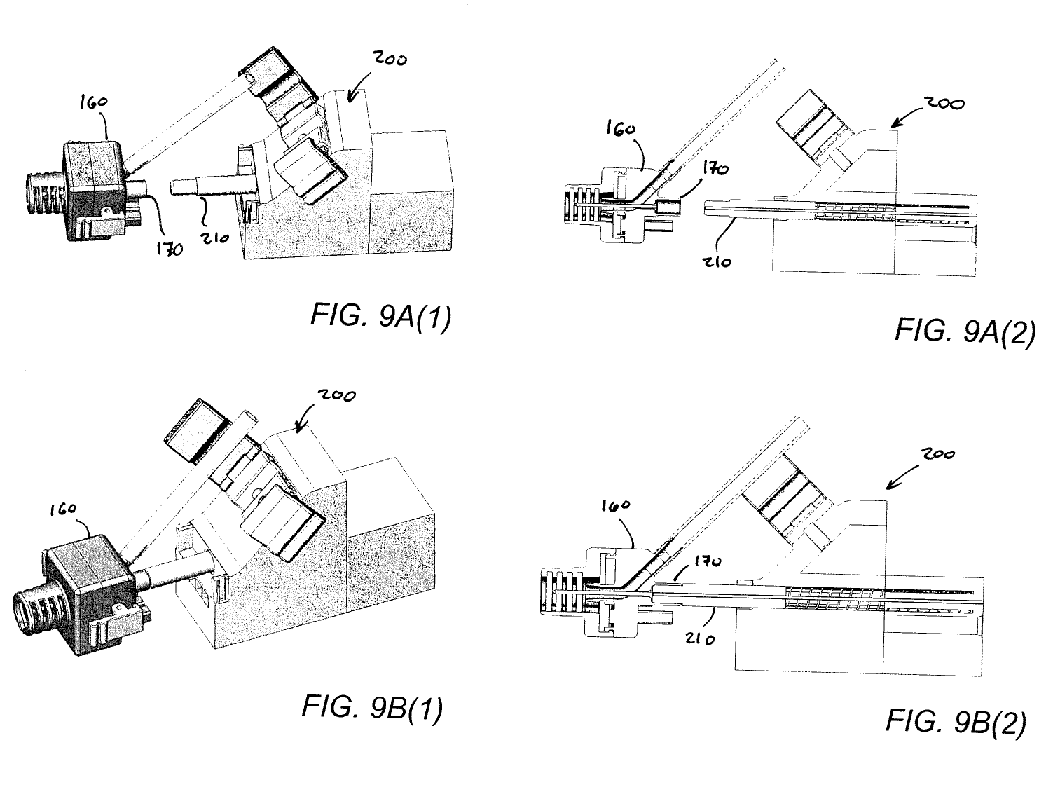

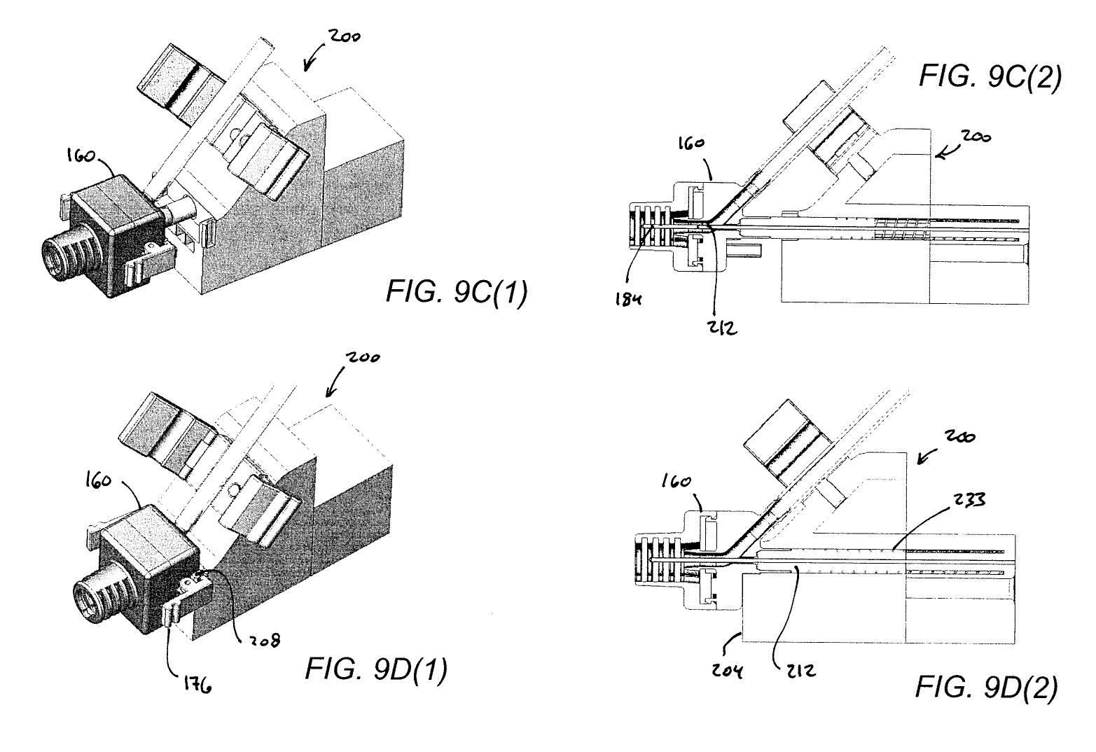

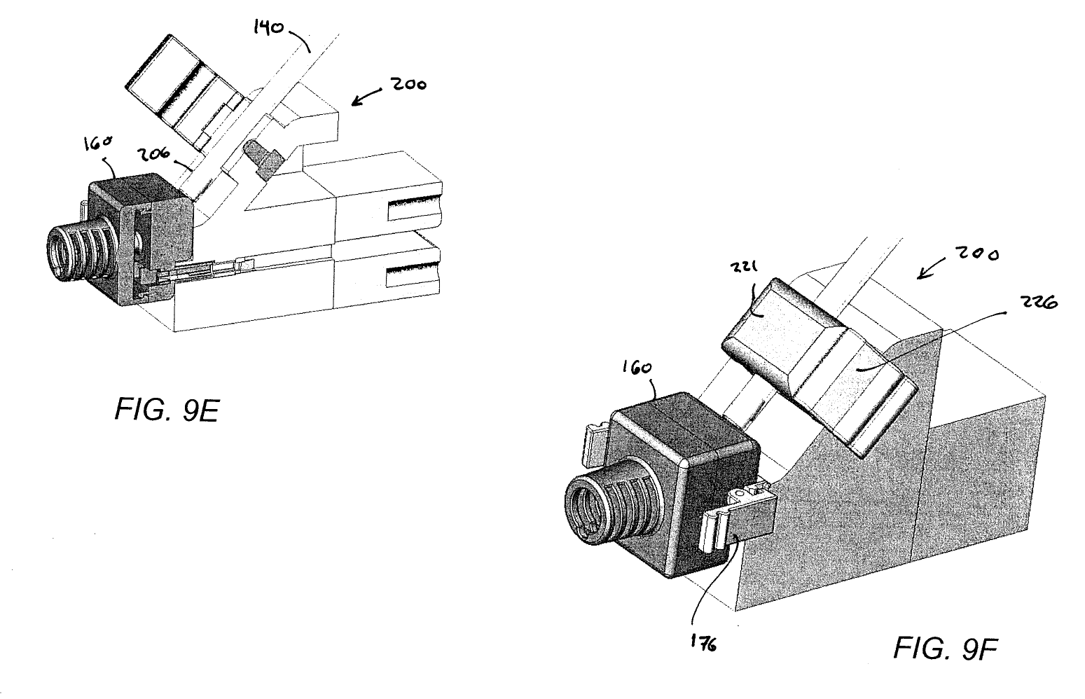

An alignment sequence is illustrated in perspective view in FIG. 9A(1), FIG. 9B(1), FIG. 9C(1), FIG. 9D(1). The same alignment sequence is illustrated in cross-sectional view in FIG. 9F and FIG. 9A(2), FIG. 9B(2), FIG. 9C(2), FIG. 9D(2). In a pre-alignment phase, shown in FIG. 9A(1) and FIG. 9A(2), the tube termination assembly 160 is positioned with respect to the controller box adapter module 200, such that the alignment/support collar 170 is approximately aligned with a proximal end of the fully extended, spring-loaded retractor pin 210. In a fine-alignment phase, shown in FIG. 9B(1) and FIG. 9B(2), the self aligning tip 214 of the spring-loaded retractor pin 210 is positioned fully within the open end of the alignment/support collar 170.

Once fine-alignment is achieved, the tube termination assembly 160 is urged toward the controller box adapter module 200 as shown in FIG. 9C(1) and FIG. 9C(2). This motion causes the spring-loaded retractor pin 210 to retract within the housing 202, further compressing the spring 234 and exposing a proximal end of the permanent flow-in temperature sensor 212. Alignment of the self-aligning tip 214 within the open end of the alignment/support collar 170 ensures alignment of proximal end of the flow-in temperature sensor 212 with an open end of the sheath 184. Being so aligned, the temperature sensor 212 can be advanced into the open ended sheath 184. When a distal end of the tube termination assembly 160 reaches the abutting surface 204, the spring loaded retractor pin 210 is fully recessed within the cavity 233. Likewise, the flow-in temperature sensor 212 is fully extended into the sheath 184 such that a proximal end portion of the temperature sensor 212 resides within a distal end of the fluid delivery tube 122 allowing for temperature measurements at the fluid input end of the tube 122. Each of the pivotal latches 176 engages a respective mating surface 208 to retain abutting engagement of the two modules 160, 200. A perspective cross sectional view of the abutted pair 160, 200 is shown in FIG. 9E. In a final step of the mating sequence shown in FIG. 9F, the fluid inlet tube retaining door 221 is closed and latched securely retaining the fluid inlet tube 140 against the fluid inlet tube retaining assembly 216.

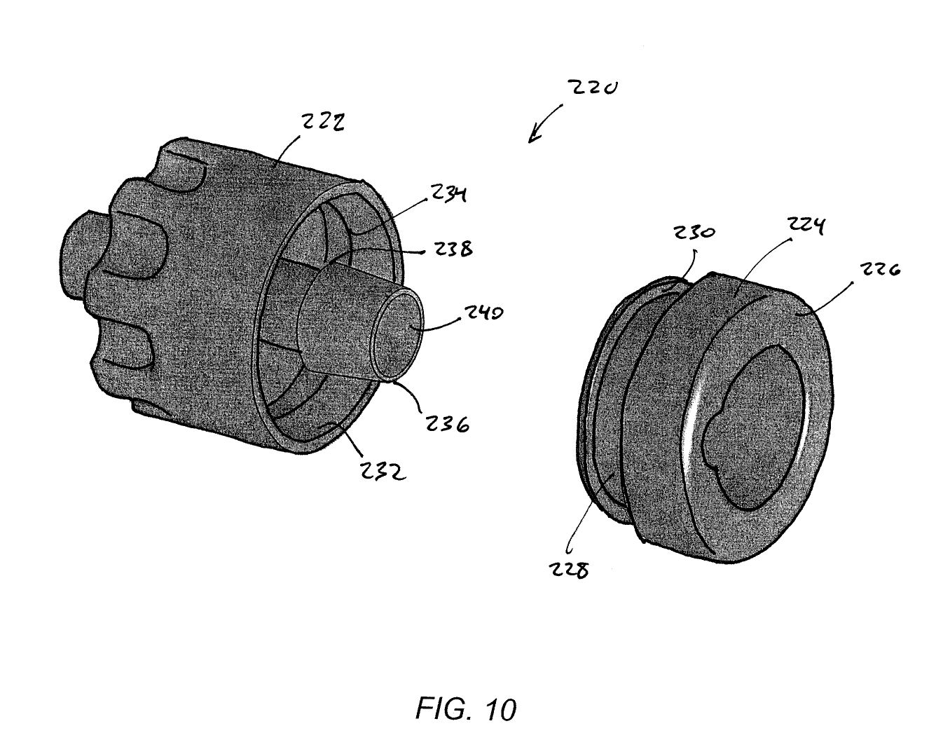

As shown in FIG. 10, an exemplary embodiment of a patient-proximal luer lock assembly 220 includes a luer lock fitting 222 and a fluid delivery tube termination 224. The fluid delivery line termination 224 includes a ferrule 226 dimensioned to fit over an outer end surface of the fluid delivery tube 122. The fluid delivery tube termination 224 includes a patient-proximal cylindrical extension 228 having an external diameter narrower than the ferrule 226. A circumferential retaining ridge 230 is defined at the proximal end of the cylindrical extension 228. The retaining ridge 230 includes an end sloping surface and a retaining surface. In some embodiments, the retaining ridge 230 extends about the entire circumference as a ring. In other embodiments, the retaining ridge 230 includes one or more circumferential openings or gaps. Such gaps can promote flexure of the retaining ridge 230 during a mating process with the luer lock fitting 222.

The luer lock fitting 222 includes an outer sleeve or collar 232 at an end adjacent to the fluid delivery tube termination 224. The collar 232 includes circumferential retaining surface 234 along an interior surface. The collar is dimensioned to accept within it the cylindrical extension 228 of the fluid delivery tube termination 224. The retaining surface 234 is positioned to abut a retaining surface of the retaining ridge 230 when the cylindrical extension 228 is fully inserted within the collar 232. The luer lock fitting 222 also includes a rigid tube adapter 236 axially positioned in a coaxial relationship with the collar 232. The rigid tube adapter 236 defines a distal end of an axial fluid channel 240 through the luer lock assembly 220. The rigid tube adapter 236 also includes a feature, such as a barbed end portion 238 to frictionally engage an interior surface of a proximal end of a compliant fluid delivery tube 122.

In some embodiments, a radial wall forming a base of the cavity defined by the collar 232 and positioned proximal to the retaining surface 234 prevents further proximal movement of the cylindrical extension 238. Together with interlocking engagement of the retaining surface 234 and the retaining ridge 230, the radial wall restricts relative axial displacement of the luer lock fitting 222 and the fluid-delivery tube termination 224, while allowing the two assembly components 222, 224 to rotate with respect to each other. Alternatively or in addition, abutment of an outer circumferential ridge 235 formed along a proximal end of the ferrule 226 and an outer circumferential ridge 237 formed along a distal end of the collar 232 limits relative axial displacement of the two assembly components 222, 224 when they are interlocked by engagement of the retaining surface 234 and the retaining ridge 230.

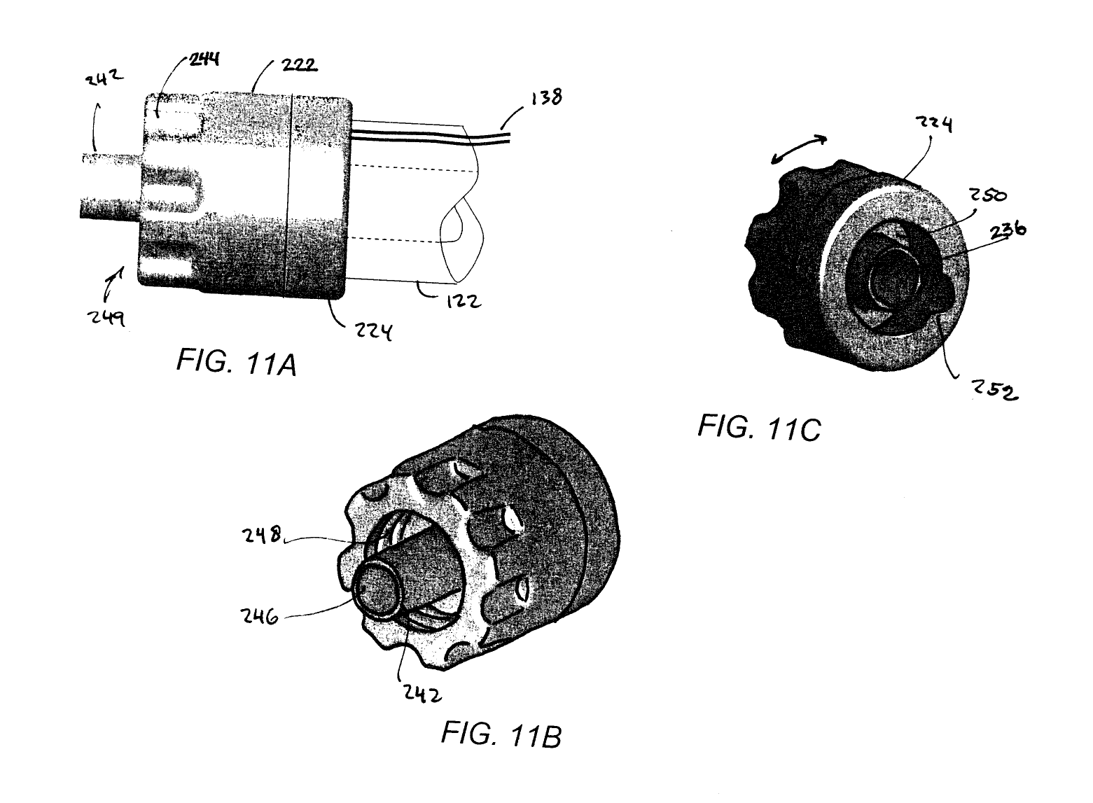

As shown in FIG. 11A, FIG. 11B, and FIG. 11C, the luer lock fitting 222 and the fluid delivery line termination 224 mate in interlocking engagement to form the luer lock assembly 220. The assembly 220 is configured to terminate a patient-proximal end of the fluid delivery tube 122 as shown. The luer lock fitting 122 includes a proximal luer lock interface 249. The luer lock interface includes a tapered central cone 242 including a central fluid channel 246 forming a proximal end of the axial fluid channel 240 through the assembly 220. An interior luer lock thread 248 is defined along an interior surface of a proximal extension of the luer lock fitting 122. The resulting arrangement can be referred to as a cooperative threaded male luer lock configured to reversibly couple with a threaded female luer lock connector (not shown) and forming a fluid-tight arrangement therebetween along the axial fluid channel 240. In some embodiments, a friction or grip feature 244, such as a circumferential arrangement of grooves is provided along at least a portion of an exterior surface of the luer lock fitting 222 to facilitate manipulation (i.e., rotation) during mating and unmating of the luer lock assembly 220. In some embodiments, a lead wire access notch 252 is formed within an interior surface of the ferrule 226 to accommodate routing of flow-out temperature sensor lead wires 138 from a proximal end of the fluid delivery tube 122.

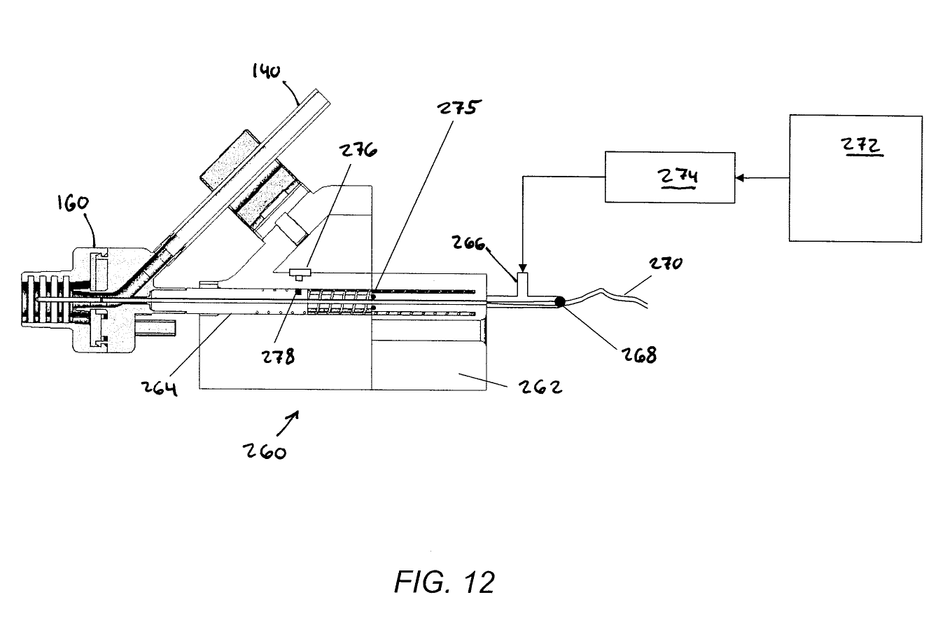

As shown in FIG. 12, a cross-sectional elevation view of the fluid-delivery line termination assembly being coupled to an alternative embodiment of a controller box adapter module includes a safety feature to detect breach of the sensor sheath 184, which could lead to contamination of the fluid. A housing 262, similar to the housing 202 described above in relation to FIG. 6, is adapted to reversibly mate with a patient-distal fluid delivery line termination 160 as described above. The housing 262 includes a cavity in fluid communication with an interior surface of the sensor sheath 184. The housing 262 also includes an air port 266 in fluid communication with the cavity through which a gas can be injected to pressurize the cavity. In order to maintain a positive pressure, all openings and joints are provided with a fluid-tight seal. For example, an O-ring seal 275 is provided at the housing-retractor pin interface. When the fluid delivery tube termination 160 is engaged with the controller box adapter module 200, an internal end of the retractor pin 210 seats against the O-ring seal 275, thereby forming a closed path from the air compressor 272 to an inside diameter of the open end of the sensor sheath 184. An air seal 268, such as an epoxy is also provided at the exit location of the flow-in temperature sensor leads 270.

An air compressor 272 is coupled to the air port 266 to provide the desired pressurization. In other embodiments, a pre-charged gas canister can be coupled to the air port 266 to accomplish the same. In either instance, a pressure regulator and/or flow valve may be included (not shown) to control the flow and pressure value. A pressure sensor 274 is provided in communication with the pressurized cavity and configured to detect a drop in pressure below a minimum pressure threshold. The pressure sensor 274 can be coupled to an alarm that may include and audio and/or visual indicator. For example, the pressure sensor 274 is in communication with the controller 128, which is configured to detect an alarm condition and indicate such to an operator or clinician.

In some embodiments, the housing includes a sensor to detect retraction of the spring-loaded retractor pin 210. Upon detecting that the retractor pin 210 has been retracted, the air compressor 272 is activated to pressurize the sheath 148. For example, the sensor can include a hall effect sensor 276 attached to the housing and a magnet fixedly attached to an interior portion of the retractor pin 210. In an extended position of the retractor pin 210, the magnet is aligned with the hall effect sensor 276, which produces an output used to deactivate the air compressor 272. Upon retraction of the retractor pin 210, the magnet is moved away from the hall effect sensor 276, which produces a changed output used to activate the air compressor 272. Alternatively or in addition, pressurization can be activated by continuity detection of one or more of the heater and flow out temperature sensor terminals 238 as the fluid delivery line termination assembly 160 is engaged with the controller box adapter module 200.

In some embodiments, a positive pressure is maintained within the sheath between a pressure threshold of about 0.2 and 2.0 psi. The hall effect sensor 276 can also be used to active the pressure sensor 274 when the retractor pin 210 has been retracted. An electrical output from the pressure sensor 274 can be used to trigger an alarm when the pressure exceeds a boundary of the pressure threshold with the retractor pin 210 retracted. Beneficially, the positive pressure can also be used to slightly expand the sheath 184 and reduce friction between an interior surface of the sheath 184 and the permanent flow in temperature sensor 212.

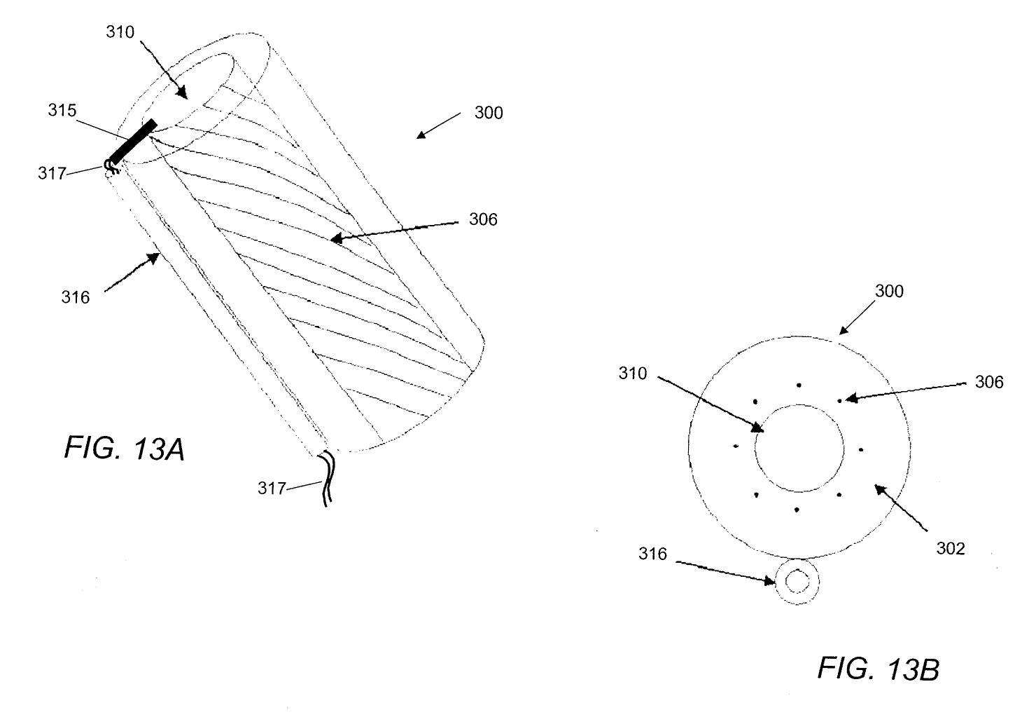

FIGS. 13A and 13B illustrate a detailed view and a cross section of a main heat tube component 300 according to one embodiment of the invention. The main heat tube component 300 may be constructed from typical medical grade tubing material 302, polyvinylchloride (PVC) or silicone for example, and a thermal element, such as resistance wire 306, which produce heat with an applied electrical current, are integrated during the tube manufacturing process. The resistance wire 306, a pure copper or copper and nickel alloy for example, function in conjunction with a variable power source as a precise and responsive heat element. The main heat tube 300 includes a sterile fluid pathway 310 for fluid which are warmed therein. Positioned within the fluid pathway are one or more thermal sensors 315, such as thermistors, which are thermally sensitive resistors that function as solid state, electronic devices for detecting thermal environmental changes. Each thermal sensor 315 is used to directly sense and report to the controller, via connection lines 317, the temperature of a fluid within the fluid pathway 310 that is proximate to the sensor 315 at the specific time. One or more thermal sensors 315 may also be used to sense and report the temperature of the fluid delivery tube 300 at specific locations and times. This temperature may then be directly related to the temperature of fluid within the fluid delivery tube 300. Another component of the fluid delivery line system 300, a sensor connection line conduit 316 is incorporated in the fluid delivery line system 300 to protect thermal sensor connection lines 317, which may extend for part of or the entire length of the main heat tube 300.

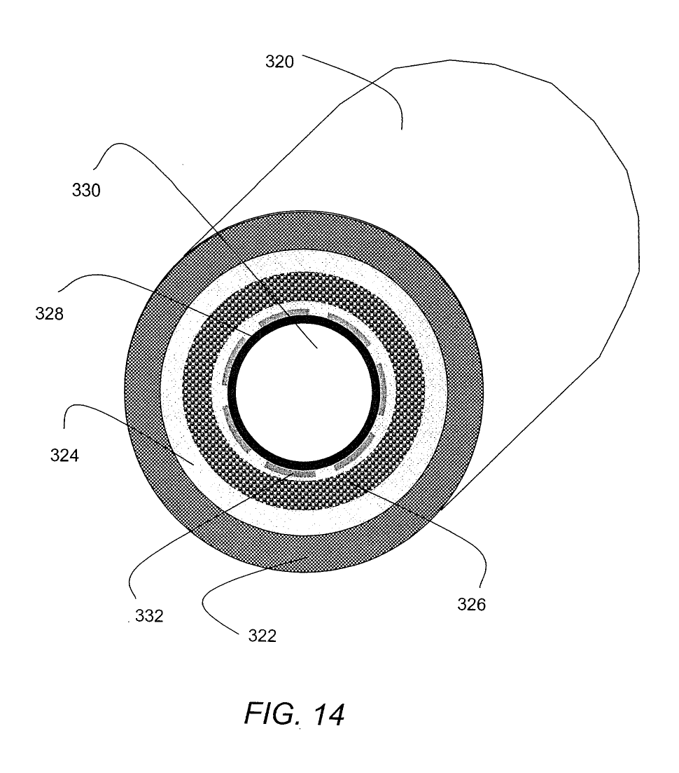

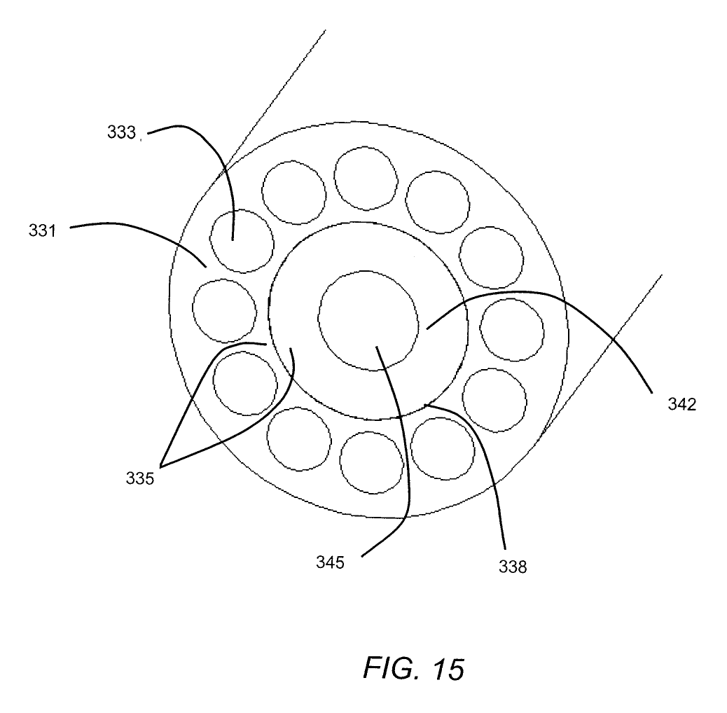

As shown in FIG. 14, which illustrates a cross section of a fluid delivery-line 320 according to one embodiment of the invention, the fluid delivery-line may include the following components. An outer sleeve or tube of an insulation material (for example) 322 surrounds a thermal medium 324. Within the thermal medium a heating element 326 is provided which may surround a fluid delivery tube 328. The fluid delivery tube includes a sterile fluid pathway 330 for fluids which are warmed therein.

Positioned adjacent the wall of the fluid delivery tube is one or more thermal sensors 332. In this embodiment, the one or more thermal sensors sense a temperature of the fluid delivery tube. This temperature may be directly related to the temperature of the fluid within the fluid delivery tube. One or more thermal sensors, e.g., wire or probe-type sensors, may also be used to directly sense the fluid within the delivery tube via direct contact.

The outer sleeve may be constructed from any tubular form of application appropriate insulation material. Such material may include plastic and foam based materials made from, for example, polyethylene. The outer sleeve may also contain or be constructed from additional materials, such as silicon rubber or urethane formulations or custom blended thermoplastics, such as those used in TYGON tubing, commercially available from Saint-Gobain Performance Plastics Corp. of Wayne, N.J. In another embodiment of the present invention, the outer sleeve component is constructed from a material that does not have properties of insulation. Where the outer sleeve is not constructed from material that has properties of insulation, the insulative function may be served by another components within the assembly.

The thermal medium may include a gas, liquid or solid, or a combination thereof, which allows heat produced by the heating element to be distributed more evenly. This is preferred since a direct application of the heat generated by the heating element to the wall of the inner tube, if the heating element is placed close to the wall of the inner fluid delivery tube, can damage or destroy the fluid being delivered by the system to the body of a patient (e.g., blood cells) since the amount of heat at the heating element may generally be higher.

Examples of the thermal medium may include air, water, saline and/or alcohol based solutions. Preferably, the thermal medium may also include ceramics, metals, plastics, natural fibers or some combination thereof. In some embodiments of the present invention, the thermal medium may be incorporated into the wall of the inner fluid delivery tube. In such an embodiment, the heating element may be positioned on the outer surface of the inner tube. The thermal medium wall thus evenly distributes the heat from the heating element to the non-heated portions of the inner fluid delivery tube and subsequently the fluid within the tube.

As shown in FIG. 15, which illustrates a cross-section of a fluid delivery-line according to some embodiments of the invention, the fluid delivery line my include the following components. A multi-lumen outer sleeve 331, in which each lumen 333 serves to contain a material, air for example, whose physical properties features both electrical and thermal insulation is a component thereof. The lumen 330 may also contain materials to assist with fluid heating or cooling functions. In some embodiments of the invention, in addition to an insulating material, e.g., air, the one or more lumens 330 contain cuts. The multi-lumen outer sleeve 331 surrounds a thermal medium 335 and as shown in the figure, the components may be manufactured as an integral unit, of identical or dissimilar materials, using known fabrication techniques such as co-extrusion or molding. Within the thermal medium, one or more heating elements 338 are provided to surround a fluid delivery tube 342. In this embodiment, the fluid delivery tube component is also manufactured integral to the thermal medium and hence outer sleeve. The fluid delivery tube includes a sterile fluid pathway 345 for fluid warmed therein.

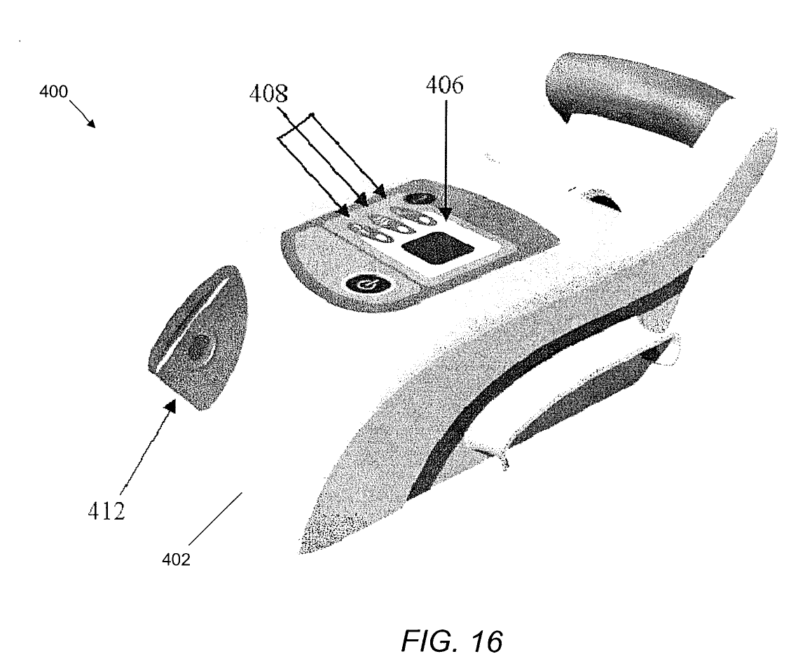

One embodiment of a controller 400, as shown in FIG. 16, may include a housing 402 made of plastic or other similar material, which contains and protects the control circuitry that regulates the electrical current to a thermal transfer source, such as a heat element, and monitors the temperature of the fluid within the fluid delivery tube. The control circuitry contains and executes a continuous control/feedback logic program with unique heat balance algorithm, which calculates discrete power input change based upon the temperature of the fluid in the delivery line at two or more locations. The controller may also include a battery pack or other power source (external or internal), a temperature display 406 for indicating a temperature of the fluid within the inner fluid delivery tube, and one or more LED lights 408. The LEDs may be used to indicate any one of the following: power level of the power source, whether the controller is connected to the heating element and/or thermal sensor, indicator light for a temperature within a prescribed range (e.g., for delivery to a patient, too hot and/or too cold). The controller may also include a speaker for audio signals.

The controller may also include a permanently mounted thermal sensor, positioned such that it is allowed direct contact with the lead tube component of the fluid delivery line. A tube clip, clamp, or recessed feature in the housing may also be included in the controller to accommodate the positioning of the lead tube adjacent to the permanent thermal sensor. The controller may also included a connector port 412 facilitates the connection of the controller to the corresponding connector for the heating element(s) and thermal sensor(s) of the fluid delivery line. These connectors may include a locking feature, which insures that connections do not come apart and/or that the connectors are fully connected.

One of skill in the art will appreciate that the one or more of the various circuits/circuitry of the controller of some of the embodiments of the present invention may be analog or digital. To that end, upon the controller including digital circuitry, for example, the controller may include a microprocessor, having memory (which may be a detachable memory module), which communicates to heating and/or thermal sensing circuitry. A power source may also be provided internal or external to the controller. LED circuitry and/or display, audio circuitry and/or output and a temperature circuitry and/or display may also be provided, each of which may communicate with the microprocessor. Additional thermal sensors for detecting the temperature of the fluid delivery line may be provided. Controls may also be included which may be used to set them temperature for the fluid (to be heated to, for example), or for setting different parameters of the controller. A serial port or USB port, for example (which may be any type of communication port familiar to one of skill in the art), may be included which allows the controller to communicate with a computer. Such communication may then be used to perform calibration tests, for example, and download heating information for heating particular types of fluids.

The temperature display may be used to display a visual indicator of the temperature e.g., an actual digital display of the temperature of the fluid. The LEDs may be used to monitor the temperature as well, and may also be used to indicate certain conditions of the controller and/or fluid delivery line. For example, the LEDs may indicate that the controller is on or off, that the temperature of the fluid has reached a predetermined value, that current is being sent to the heating element, and the like. The audio circuitry/output may be used to provide audio indication that fluid has reached a desired temperature, for example.

The controller may also include digital/analog conversion circuits for operating the heating element and collecting temperature information from the one or more thermal sensors. Moreover, in some embodiments of the present invention, one or more (or all) functions of the controller may be replaced by a computer (desktop, mini/micro, mainframe, PDA and the like), having connectors and corresponding circuitry to carry out the application and control of current to the heating element, the sensing of temperature, and/or the actuation of a valve for controlling the flow of fluid through the fluid delivery line of the present invention.

The controller may include other features such as a variable temperature selector for changing a resultant temperature of the fluid within the inner fluid delivery line. Thus, if, for example, a patient is suffering from hypothermia, a medicating fluid (e.g., one to aid in the recovery of the patient) may be kept at a temperature above the body temperature of the patient, but below normal. Accordingly, the heating and thermal sensing circuitry may include circuitry for gradually increasing a resultant temperature of the fluid within the fluid delivery tube to aid the recovery of a hypothermia patient. In that regard, the heating and thermal sensing circuitry may include circuitry for gradual increase or decrease of a resultant temperature of the fluid within the fluid delivery tube for any number of therapeutic reasons. Of course, a range of temperatures within which the controller and present system may operate may be, for example, between 32 degrees F. and 105 degrees F.

The controller may also include circuitry for actuating valve. Such circuitry may be integral or connected to the heating and thermal sensing circuitry such that upon the thermal sensing circuitry detecting the resultant temperature of the fluid within the inner fluid delivery tube being at a predetermined temperature, the circuitry actuates the valve to allow the fluid to flow into the patient. Accordingly, the circuitry may be connected to the valve via a wire, which sends current to an electro-mechanical actuator at the valve.

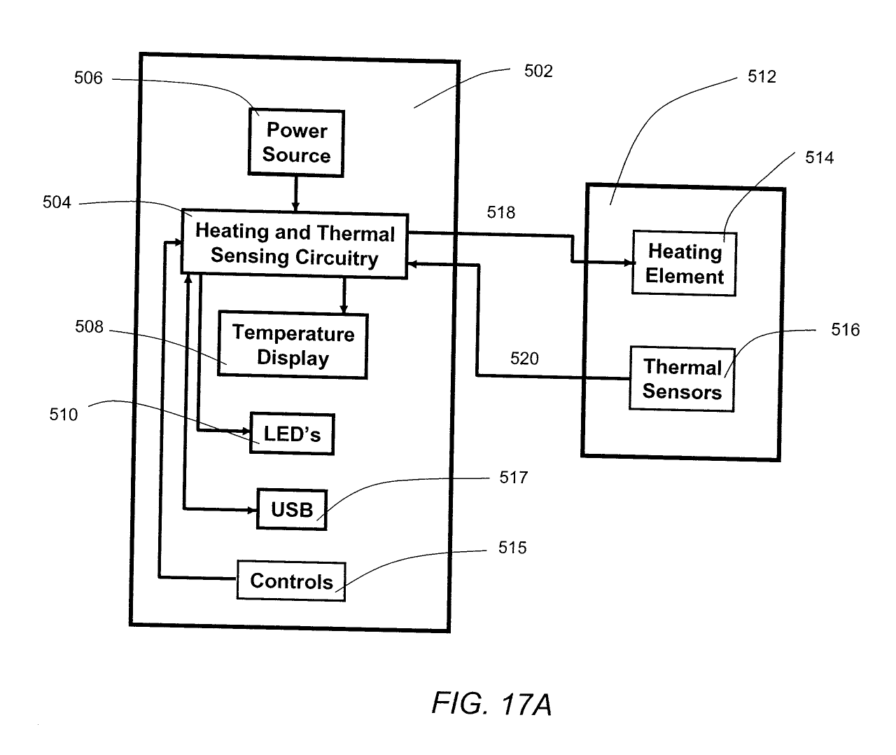

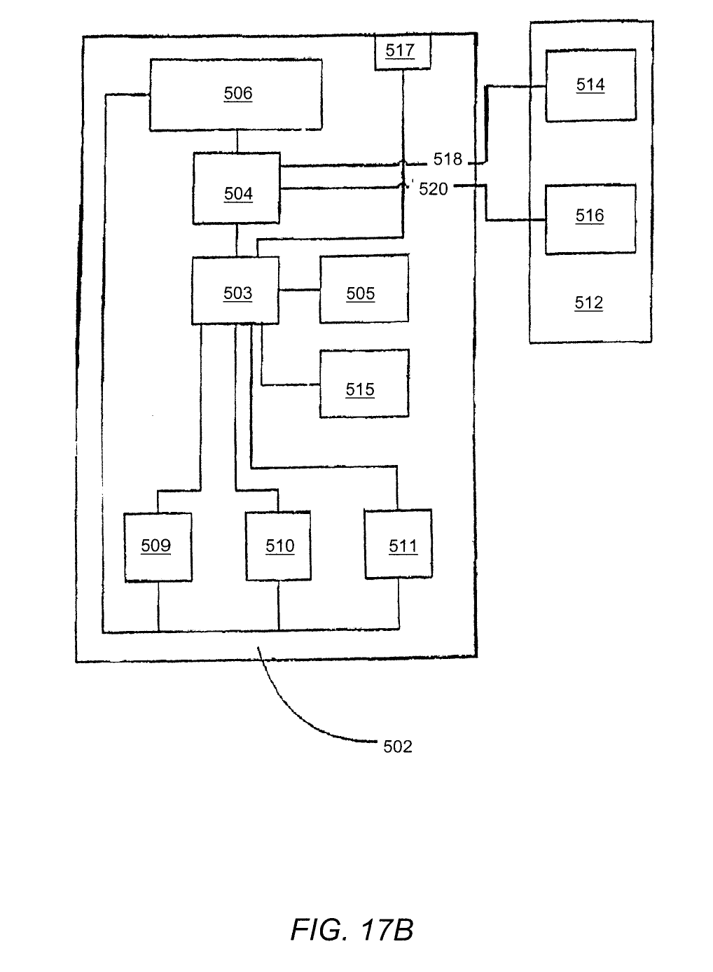

As shown in FIG. 17A, in one embodiment, upon the controller including digital circuitry, for example, the controller 502 may include a heating and/or thermal sensing circuitry 504. A power source 506 may also be provided internal or external to the controller. A temperature display 508, LED circuitry 510, a communication port, e.g., USB port 517, and controls 515 may also be provided. The heating and sensing circuitry 504 may be connected to the heating element(s) 514 and thermal sensor(s) 516 of the fluid delivery-line 512 via connections 518 and 520, respectively.

As illustrated in FIG. 17B, in another embodiment, upon the controller including digital circuitry, for example, the controller may include a microprocessor 503, having memory 505 (which may be a detachable memory module), which communicates to heating and/or thermal sensing circuitry 504. A power source 506 may also be provided internal or external to the controller. LED circuitry and/or display 509, audio circuitry and/or output 510 and a temperature circuitry and/or display 511 may also be provided, each of which may communicate with the microprocessor. The heating and sensing circuitry 504 may be connected to the heating element(s) 514 and thermal sensor(s) 516 via connections 518 and 520, respectively.

Controls 515 may also be included which may be used to set them temperature for the fluid (to be heated to, for example), or for setting different parameters of the controller. For example, the memory may include heating routines for a specific type of fluid. Using controls 515, a user can then select an appropriate heating routine.

A serial port or USB port 517, for example (which may be any type of communication port familiar to one of skill in the art), may be included which allows the controller to communicate with a computer. Such communication may then be used to perform calibration tests, for example, and download heating information for heating particular types of fluids.

The temperature display may be used to display a visual indicator of the temperature, e.g., an actual digital display of the temperature of the fluid. The LEDs may be used to monitor the temperature as well, and may also be used to indicate certain conditions of the controller and/or fluid delivery-line. For example, the LEDs may indicate that the controller is on or off, that the temperature of the fluid has reached a predetermined value, that current is being sent to the heating element, and the like. The audio circuitry/output may be used to provide audio indication that fluid has reached a desired temperature, for example.

The controller may also include digital/analog conversion circuits for operating the heating element and collecting temperature information from the one or more thermal sensors. Moreover, in some embodiments of the present invention, one or more (or all) functions of the controller may be replaced by a computer (desktop, mini/micro, mainframe, PDA and the like), having connectors and corresponding circuitry to carry out the application and control of current to the heating element, the sensing of temperature, and/or the actuation of a valve for controlling the flow of fluid through the fluid delivery-line of the present invention.

The controller may include other features such as a variable temperature selector for changing a resultant temperature of the fluid within the inner fluid delivery-line 615. Thus, if, for example, a patient is suffering from hypothermia, a medicating fluid (e.g., to aid in the recovery of the patient) may be kept at a temperature above the body temperature of the patient, but below normal. Accordingly, the heating and thermal sensing circuitry may include circuitry for gradually increasing a resultant temperature of the fluid within the fluid delivery tube to aid the recovery of a hypothermia patient. In that regard, the heating and thermal sensing circuitry may include circuitry for gradual increase or decrease of a resultant temperature of the fluid within the fluid delivery tube for any number of therapeutic reasons. Of course, a range of temperatures within which the controller and present system may operate may be, e.g., between 32° F. and 105° F.

The controller may also include circuitry for actuating valve 112 (FIG. 1). Such circuitry may be integral or connected to the heating and thermal sensing circuitry such that upon the thermal sensing circuitry detecting the resultant temperature of the fluid within the inner fluid delivery tube being at a predetermined temperature, the circuitry actuates the valve to allow the fluid to flow into the patient. Accordingly, the circuitry may be connected to the valve via a wire, which sends current to an electro-mechanical actuator at the valve.

In some circumstances, patients may require pre or post-operative cooling for a variety of reasons, including, for example, treatment of a malignant hypothermia crisis and induction of therapeutic hypothermia for neurosurgery.

It is within the scope of the present invention that the system of the present invention can be used for cooling a fluid. In one embodiment, the heat element is replaced with a hollow tube for circulating a coolant or a solid metallic chilling element that serves to lower the temperature of the fluid in the delivery-line. This configuration may be used in the delivery of cooled fluid to a patient, for IV use and/or other fluid administration techniques.

In another aspect of the present invention, the system has both heating and cooling elements and can be used for warming and cooling, thereby controlling the temperature of a fluid, the temperature of a target tissue, or the temperature of a patient.

The present system may be used with any types of power sources, e.g., AC, DC, wall outlet jack, batteries, vehicle power systems. The present system may be mounted on, or supported by, any type of support structure, e.g., wall, cart, table, floor. The systems preferably heat or cool items to desired temperatures within the approximate range of 70° F. to 150° F. In some embodiments, the power source provides a fixed power output to within a regulated tolerance. A power varying circuit is coupled between the power source and the controller and/or thermal transfer elements to control the amount of power applied to the system. Such power varying circuits are generally know, such as dimmer circuits, and can include one or more of pulse-width modulators, varistors, resistor divider networks, transformers, and variacs.

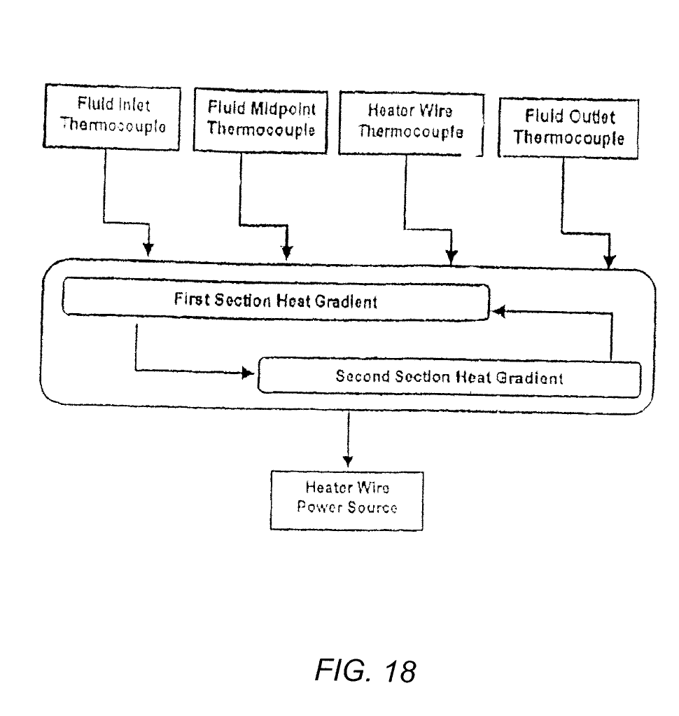

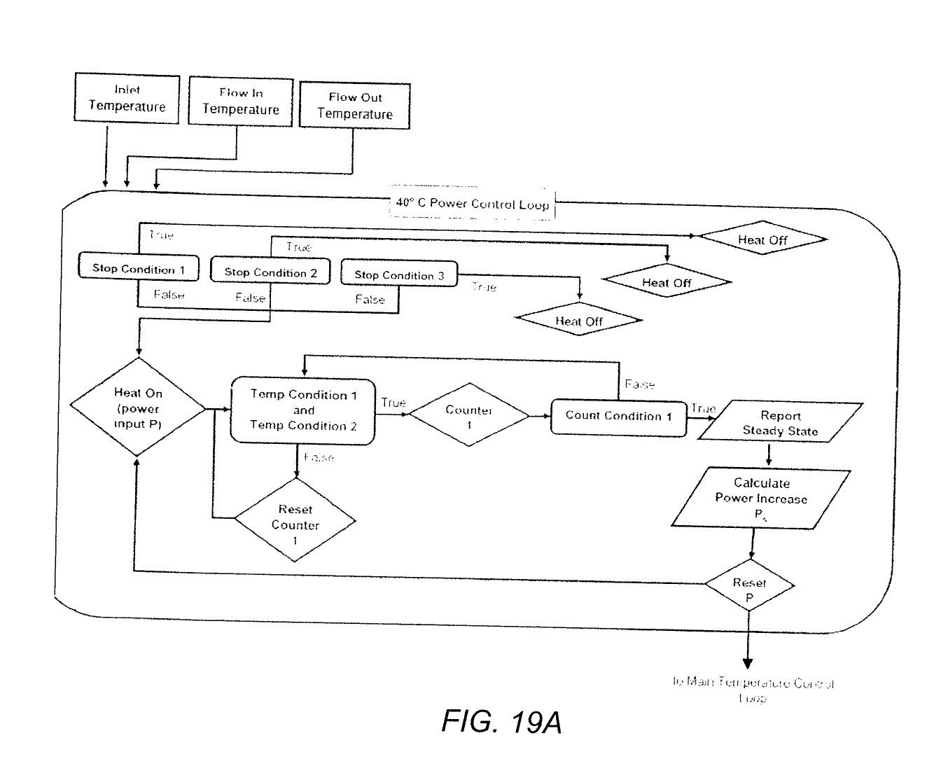

For constant flow rate the first section, defined by the inlet to midpoint, is used to modify the heat input such that the second section, defined by the midpoint to outlet, is used to generate the desired output for the entire length of the tube. In turn, the temperature at the outlet combined with the temperature at the midpoint provide actual data for comparison to the expected temperatures based on the revised heat input derived from the inlet and midpoint temperatures. Also, each thermocouple (inlet, midpoint and outlet) provides point temperature valves, which when coupled with the heater wire data, are used to determine changes in flow rate. The diagram shown in FIG. 18 illustrates the flow of data and controls for this process. The final objective of the algorithm is to iterate the heat input based on the temperature gradient from the thermocouples, such that the desired output temperature is achieved for the given fluid with minimum amount of required heat input. The reason for doing so is to maximize high flow rate capability without creating a fluid overheating condition when flow is stopped abruptly.

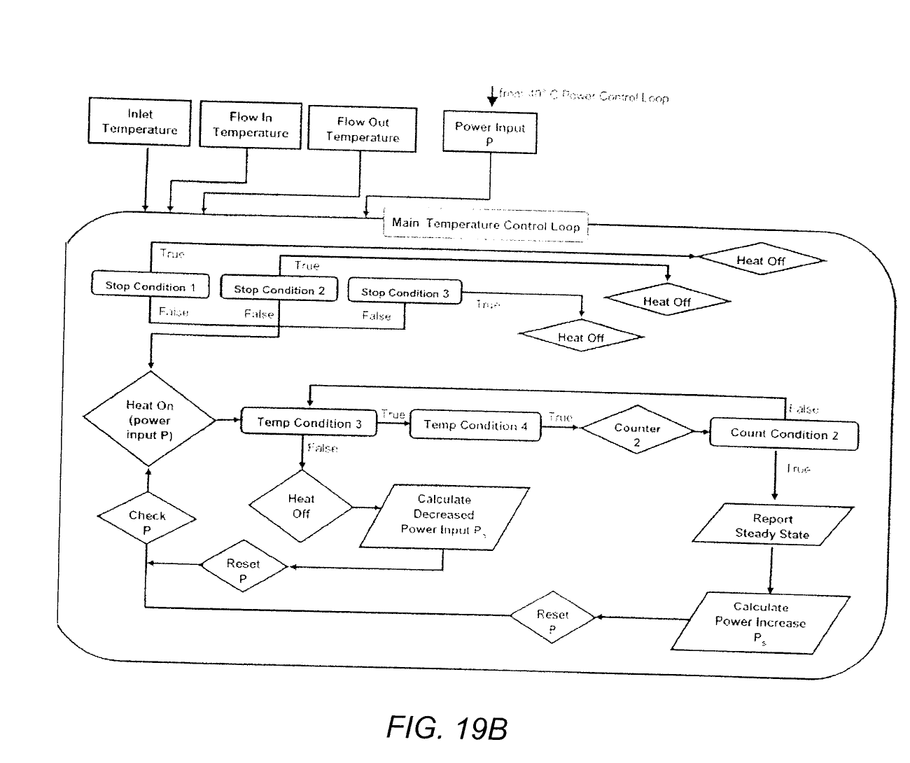

As shown in Table 1, FIG. 19A and FIG. 19B, a flow diagram of the functions of the embedded control/feedback logic program of the controller utilizes two main program routines. The first routine uses the data from the two thermal sensors at the inlet and outlet of the heating assembly and the one thermal sensor on the exterior of the fluid supply line to determine the amount of heating for a know power input. This routine utilizes an algorithm developed specifically for this application that generates a required power input based upon the individual flow rate and temperature values for each fluid transfused. Once the fluid temperature reaches a steady state and the power input increase is calculated, the first routine initiates the second routine. The second routine uses data from the same three thermal sensors and from the first routine to achieve the desired fluid temperature and maintain said fluid temperature, despite changes in flow rate. This routine utilizes the specifically developed algorithm that generates a required power input based upon the individual flow rate and temperature values for each fluid transfused. In addition the second routine performs checks and calculations for power decrease event, stop flow or drastic flow reduction for example. The second loop is optimized for extended fluid temperature management using variable power input. The logic developed and described here is based on know parameters such as heating element resistance and initial power input.

TABLE 1
User Defined Software Set Points

| Descriptor | Name | Value (range) |
|---|---|---|
| Stop Condition 1: Flow In Temperature (T2) set | flowin max | 30° C. (30-35) |
| point value | | |
| Stop Condition 2: Inlet Temperature (T1) - Flow In | flowin-inlet | 1° (0.1-2.0) |
| Temperature (T2) set point value | | |
| Stop Condition 3: Flow Out Temperature (T3) set | flowout max | 40° C. (40-42) |
| point value | | |
| Counter 1: count initial value | preset start | 1 |
| | value | |
| Count Condition 1: count variable (n) set point value | preset stop | 10 |
| | value | |
| Stop Condition 3: Flow In Temperature (T2) set | flowin max | 30° C. (30-35) |
| point value | | |
| Stop Condition 4: Inlet Temperature (T1) - Flow In | flowin-inlet | 1° (0.1-2.0) |
| Temperature (T2) set point value | | |
| Stop Condition 5: Flow Out Temperature (T3) set | flowout max | 40° C. (40-42) |
| point value | | |
| Counter 2: count initial value | preset start | 1 |
| | value | |
| Count Condition 2: count variable (n) set point value | preset stop | 10 |
| | value | |
| flowin adder | C_{fa} | 3° C. (1-10) |
| temperature reduction divisor | Cₜᵣ | 10 |

Having now described a few embodiments of the invention, it should be apparent to those skilled in the art that the foregoing is merely illustrative and not limiting, having been presented by way of example only. Numerous modifications and other embodiments are within the scope of ordinary skill in the art and are contemplated as falling within the scope of the invention as defined by the appended claims and equivalents thereto. The contents of any references cited throughout this application are hereby incorporated by reference. The appropriate components, processes, and methods of those documents may be selected for the invention and embodiments thereof.

What is claimed is:

1. A disposable assembly for heating or cooling a fluid flow for delivery into a body of a patient comprising:
an elongated fluid transfer tube comprising a lumen extending therethrough;
a distal connector assembly attached at one end of the elongated fluid transfer tube, the distal connector assembly including:
a fluid channel in fluid communication with the lumen of the elongated fluid transfer tube;
a fitting for connecting the fluid channel to a fluid inlet tube; and
an axial shroud open at one end and extending at least partially into the fluid path, the shroud adapted to receive a thermal probe inserted at the open end, the shroud isolating the thermal probe from fluid path.

2. The disposable assembly of claim 1, further comprising at least one heating or cooling element positioned proximate a surface of the elongated tube to heat or cool fluid contained therein.

3. The disposable assembly of claim 2, wherein the distal connector assembly further comprises at least one electrical terminal in electrical communication with the at least one heating or cooling element.

4. The disposable assembly of claim 3, further comprising a thermal probe positioned to measure temperature of a fluid at an opposite end of the elongated tube, the thermal probe in electrical communication with the at least one electrical terminal.

5. The disposable assembly of claim 2, comprising more than one heating or cooling elements positioned proximate a surface of the elongated tube and spirally surrounding a substantial length thereof to heat or cool fluid contained therein and an electrical connection interconnecting distal ends of the more than one heating or cooling elements.

6. The disposable assembly of claim 1, wherein the distal connector includes at least one releasable mechanical fastener configured to reliably engage a controller box adapter assembly.

7. The disposable assembly of claim 1, further comprising a proximal connector assembly attached to an opposite end of the elongated fluid delivery tube, the proximal connector assembly including a fluid channel in fluid communication with the lumen of the elongated tube.

8. The disposable assembly of claim 7, wherein the distal connector assembly is a luer lock connector.

9. The system according to claim 1, further comprising a pressure source pressurizing the axial shroud and a pressure sensor positioned to detect a drop in pressure indicative of a leak.

10. A system for heating a fluid for delivery into a body of a patient comprising:
an elongated fluid delivery line comprising:
a tube for communicating a fluid along a fluid path;
at least one fluid connector coupled to one end of the elongated fluid delivery line; and
at least one elongated fluid-tight shroud open at one end and extending at least partially into the fluid path, the shroud adapted to receive a thermal probe at the open end.

11. The system according to claim 10, wherein the elongated fluid-tight shroud extends axially along an end portion of the fluid path.

12. The system according to claim 10, further comprising a controller.

13. The system according to claim 12, wherein the controller is connected to a power source.

14. The system according to claim 13, wherein the power source is selected from the group consisting of: a single-use battery pack, a rechargeable battery pack, AC power, and DC power.

15. The system according to claim 14, further comprising a power varying circuit to vary power supplied by the power source.

16. The system according to claim 12, further comprising at least one heating element extending along a substantial length of the tube.

17. The system according to claim 16, wherein the at least one heating element spirally surrounds the tube.

18. The system according to claim 17, wherein the at least one heating element is in electrical contact with the controller.

19. The system according to claim 10, wherein the elongated fluid delivery tube includes a transfusion needle and/or a luer lock connector at one end.

20. The system according to claim 10, wherein the tube is sterile prior to use.

21. The system according to claim 10, further comprising a pressure source pressurizing the at least one elongated fluid-tight shroud and a pressure sensor positioned to detect a drop in pressure indicative of a leak.

22. A method of heating or cooling a fluid for delivery into the body of a patient comprising:
providing an elongated fluid delivery tube having a first end for connection to a fluid source and a second end for delivering the fluid from the fluid source to a destination;
applying an electrical current to a heating or cooling element proximate to and/or within the elongated fluid delivery tube to heat fluid therein to a predetermined temperature;
positioning a reusable thermal sensor within a disposable fluid-tight shroud extending into a fluid path at one end of the fluid delivery tube;
sensing a temperature using the reusable thermal sensor, the temperature corresponding to the temperature of the fluid within the tube; and
adjusting the current applied to heating element based upon the sensed temperature to cause a change in temperature.
